(19) United States
(12) Patent Application Publication
(10) Pub. No.: US 2021/0093207 A1
(43) Pub. Date: Apr. 1, 2021

(54) DEVICE, METHOD AND PROGRAM FOR MONITORING PERFUSION OF A TISSUE

(71) Applicant: APD ADVANCED PERFUSION DIAGNOSTIC, Villeurbanne (FR)

(72) Inventors: Bernard ALLAOUCHICHE, Lyon (FR); Benjamin DELCROIX, Villeneuve-Loubet (FR); Dominique FALCON, Lyon (FR); Remi BONIDAL, Villeurbanne (FR)

(21) Appl. No.: 16/464,616

(22) PCT Filed: Nov. 28, 2017
(86) PCT No.: PCT/FR17/53279

§ 371 Date: May 28, 2019

(30) Foreign Application Priority Data
Nov. 28, 2016 (FR) 16 61555

(51) Int. Cl. A61B 5/026; A61B 5/0295; A61B 5/00; A61B 5/11

(52) U.S. Cl. CPC A61B 5/0261; A61B 5/0295; A61B 5/6853; A61B 2562/0247; A61B 5/1107; A61B 5/4255; A61B 5/6862

(57) ABSTRACT

The invention includes monitoring the perfusion of a tissue of an organ using a device having a perfusion sensor to detect a perfusion of the tissue, and at least one activity sensor to detect a muscle activity of the organ. The at least one activity sensor is arranged in proximity to the perfusion sensor. Both sensors are linked to a processing unit. The processing unit receives, as input, perfusion measurement data and muscle activity data, and allows the muscle activity data to be taken into account for monitoring the perfusion of the tissue. The perfusion measurement data are invalidated during the muscle activity periods.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to the field of measuring devices and methods for assessing the metabolic parameters relating to the perfusion of a patient's organ, in particular an organ comprising a mucosa and muscle tissues. The invention also relates to an associated monitoring method and program.

Such a device can be used in numerous applications to monitor patients prone to tissue hypoperfusion. These disorders can have various origins: hemorrhagic, infectious or inflammatory.

### 2. Description of Related Art Including Information Disclosed Under 37 CFR 1.97 and 37 CFR 1.98

Monitoring biological parameters relating to perfusion chiefly provides the user with information relating to the microcirculation of the organ in order to help make the appropriate choice of treatment for the patient. In fact, knowledge of the state of local perfusion, particularly of the digestive mucosa, is an important factor in the management of patients who may display alterations in said perfusion.

For example, the technology of photoplethysmography is a means used to evaluate the perfusion of a tissue to a known depth. Unfortunately, particularly in relation to tissues such as those of the rectum, the measurements can be distorted by the flow of fecal matter at the interface between the sensor and the wall. If this aspect is counteracted by a plug, involuntary contractions of the rectum to expel the plug will lead to a change in blood flow in the area of measurement and distort the measurements.

### BRIEF SUMMARY OF THE INVENTION

One aim of the invention is to propose a device for monitoring the perfusion of a tissue which provides measurements that are less distorted than in the prior art.

In order to achieve this aim, the invention proposes a device for monitoring the perfusion of a tissue of an organ, comprising
a perfusion sensor configured to detect a perfusion of the tissue, and
at least one activity sensor configured to detect a muscle activity of the organ, said at least one activity sensor being arranged in proximity to the perfusion sensor, said sensors being configured to be linked to a processing unit.

Advantageously, the device according to the invention enables data from a perfusion sensor to be linked to those of a muscle activity sensor in order to monitor perfusion, taking muscle activity into account, particularly during periods of contractions of the rectum to expel a plug upstream of the device.

More particularly, the processing unit receives, as input, perfusion measurement data and muscle activity data, and allows the muscle activity data to be taken into account for monitoring the perfusion of the tissue. Preferably, the measurement data are invalidated during the muscle activity periods.

According to other aspects taken in isolation or combined according to all of the technically feasible combinations:

the device comprises an occlusion section comprising the activity sensor, and a perfusion measurement section comprising the perfusion sensor; and/or

the occlusion section is movable between a folded position in which it is small in a radial direction and a deployed position in which it is large in the radial direction; and/or

the occlusion section comprises a balloon, a stent or a shape-memory material, configured to cause the occlusion section to switch between the folded position and the deployed position; and/or

the perfusion-measurement section is preferably inflatable and is movable between a folded position in which it is small in a radial direction and a deployed position in which it is large in the radial direction; and/or

the perfusion sensor is a photoplethysmographic sensor; and/or

the activity sensor is a pressure sensor, a force sensor and/or a pneumatic pressure-measurement device; and/or

the perfusion measurement section comprises a second muscle activity sensor; and/or

the occlusion section comprises a container and an evacuation channel.

The invention also relates to an apparatus for monitoring the perfusion of a tissue of an organ, comprising a device according to the invention and a processing unit linked to said sensors.

Another aim of the invention concerns a method for monitoring the perfusion of a tissue of an organ, comprising steps for:
acquiring tissue perfusion measurement data,
acquiring organ muscle activity measurement data,
monitoring the perfusion measurement data by taking into account the muscle activity data.

Preferably, the step of monitoring the perfusion measurement data by taking into account the muscle activity data includes monitoring outside the muscle activity periods, particularly beyond a threshold, and/or invalidating the perfusion measurement data during the muscle activity periods.

The invention also relates to a computer program that can be loaded onto a processing unit, comprising portions of program code for performing the steps of the method according to the invention, when said program is run on said processing unit.

### BRIEF DESCRIPTION OF THE SEVERAL VIEWS OF THE DRAWINGS

Further details of the invention will emerge from the description of non-limiting embodiments and on the basis of the accompanying Figures.

FIG. 1 is a schematic view of a diagram of a device according to the invention, inserted in a rectum.

FIG. 2 is a schematic view of the device according to a first embodiment of the invention.

FIG. 3 is a schematic view of the device according to a second embodiment of the invention.

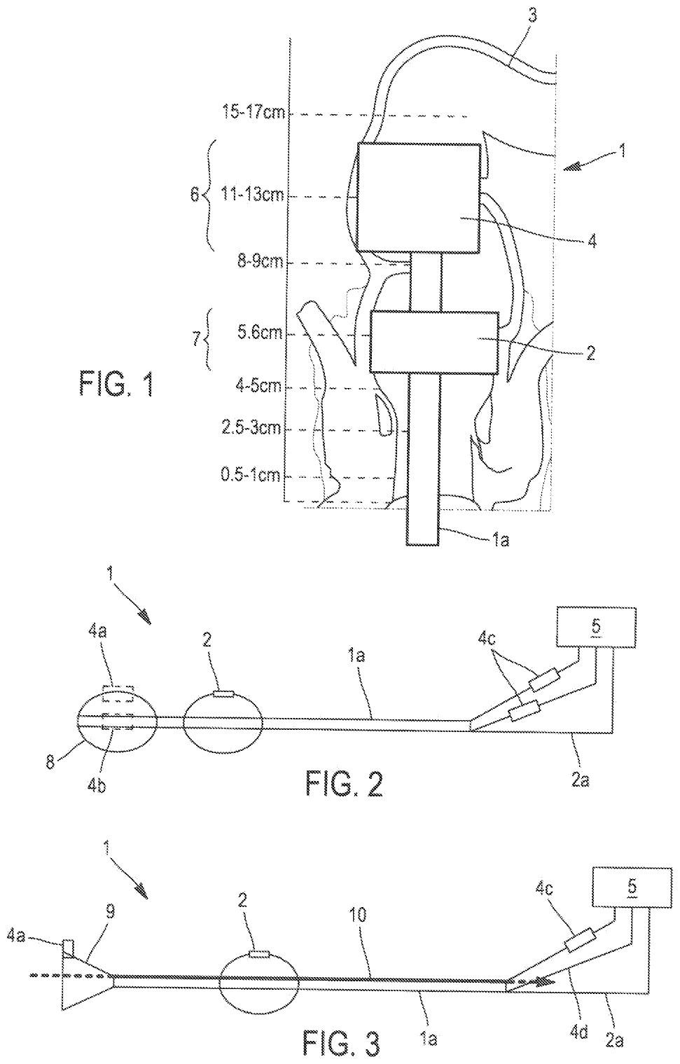

### DETAILED DESCRIPTION OF THE INVENTION

The present invention relates to a device for monitoring 1 perfusion of a tissue such as a rectal mucosa. In particular, the device 1 comprises a probe in the form of a catheter. Preferably, the body 1a of the catheter is not rigid.

Such a device 1 comprises a perfusion sensor 2 configured to detect a perfusion of the tissue. The perfusion sensor 2 is for example a sensor using photoplethysmography technology, namely a photoplethysmographic sensor. This type of sensor 2 linked to such a device 1 and the associated perfusion measurement have been described in detail specifically in applications WO2013127819 and WO2015044336, concerning measurements in a particular tissue, namely a duodenal mucosa.

In order for a photoplethysmographic sensor to be able to give a reliable indication of perfusion over time, the photon path between the transmitter and the receiver must be constant in a given application. In particular, firstly, it is important that the sensor is placed flat against the wall, secondly, that the interface between the sensor and the wall of the mucosa is always the same, and, thirdly, that the composition of the tissue remains unchanged.

In the case of both a rectal measurement and a duodenal measurement, the first point is resolved by adopting an approach of the same type as for a duodenal probe, described in the above-mentioned applications, specifically a balloon or a stent enabling the sensor to be placed flat against the wall.

As regards the second point, in the context of a measurement in another specific tissue such as a rectal mucosa 3, stools can begin to place themselves between the perfusion sensor 2 and the wall and thus distort the measurements.

As regards the third point, it poses no particular problem except in the case where the muscles surrounding the rectal ampulla contract in order to expel the stools. In this case, the mucosa is crushed between the muscles and the contents of the rectal ampulla, which results in a densification of the blood network without increasing the total flow. This densification can distort the perfusion measurements.

A first solution is to block the arrival of the stools upstream of the measurement point in order to eliminate the corruption of the measurement due to stools placing themselves between the sensor 2 and the mucosa. For example, a plug can be used.

Unfortunately, this solution is problematic in that the stools will accumulate above the plug. Beyond a certain quantity of stools, the pressure of the stools on the mucosa will exceed a threshold, estimated at 3 kPa, which will trigger the expulsion reflex.

This expulsion reflex will change the density of the capillary network resulting in a value of the perfusion indicator calculated as incorrect in the sense that it will no longer be comparable to the previous values.

It is particularly important to know when muscle activity starts so that it can be taken into account for the perfusion measurements. This is all the more important given that muscle activity can start well before the actual expulsion.

In order to overcome these drawbacks, the device according to the invention also comprises at least one activity sensor 4 configured to detect a muscle activity of the organ, said at least one activity sensor being arranged in proximity to the perfusion sensor 2. The perfusion sensor 2 and the activity sensor 4 are configured to be linked to a processing unit 5.

More particularly, the processing unit 5 receives, as input, perfusion measurement data and muscle activity data to be taken into account for monitoring the perfusion of the tissue. Preferably, the perfusion measurement data are invalidated during the muscle activity periods.

According to a variation, the monitoring device 1 comprises an occlusion section 6 comprising the activity sensor 4, and a perfusion measurement section 7 comprising the perfusion sensor 4. In particular, the occlusion section 6 is arranged upstream of the perfusion measurement section 7 with reference to the flow direction.

Advantageously, the occlusion section 6 forms part of the device and does away with the need for a plug, which would be difficult to remove after use.

In particular, the occlusion section 6 is configured so as to create a “semi-occlusion”, i.e. so that it partially blocks both the solid stools and the liquid stools, or just the solid stools; and it can be expelled by the muscles of the rectum 3.

Advantageously, the occlusion section 6 provides a measuring space around the measurement section 7 without the measurements being distorted by stools placing themselves between the sensor and the mucosa of the rectum 3.

According to a variation, the occlusion section 6 is movable between a folded position in which it is small in a radial direction and a deployed position in which it is large in the radial direction.

Advantageously, the occlusion section 6 can assume a first configuration enabling easier insertion, namely the folded position, and a second configuration enabling good semi-occlusion of the rectum, namely the deployed position.

The occlusion section 6 can be inflatable. For example it comprises a balloon. Advantageously, an inflatable occlusion section enables a simplified change of configuration by simple inflation/deflation of the occlusion section.

The occlusion section 6 can comprise a stent. Advantageously, a stent enables a rigid deployed position to be achieved.

The occlusion section 6 can comprise a shape-memory material such as a shape-memory plastic.

According to a variation, the perfusion measurement section 7 is preferably inflatable and is movable between a folded position in which it is small in a radial direction and a deployed position in which it is large in the radial direction.

The advantages of the deployable occlusion section 6 are applicable mutatis mutandis to the deployable perfusion section 7.

According to a variation, the activity sensor 4 is a force sensor 4a preferably arranged on the occlusion section 6. In particular, the force section 4a is arranged between the occlusion section 6 and the mucosa of the rectum 3. The force sensor 4a measures the mechanical pressure of the mucosa on the occlusion section 6. Advantageously, a force sensor 4a enables local measurement of the muscle activity of the mucosa.

According to a variation, the activity sensor 4 is a local pressure sensor 4b. In particular, the local pressure sensor 4b is arranged in the occlusion section 6. The local pressure sensor 4b measures the pneumatic pressure in the occlusion section 6. Advantageously, a local pressure sensor 4b enables local measurement of the muscle activity of the mucosa by measurement of the pneumatic pressure.

According to a variation, the activity sensor 4 is an external pneumatic pressure measuring device 4c. In particular, this device 4c is arranged on a section not introduced into the rectum 3. Advantageously, an external pneumatic pressure measuring device 4c limits the size of the monitoring device 1 as regards the part inserted into the rectum 3.

According to a variation, the perfusion measurement section 7 comprises a second activity sensor 4 that can be one of the activity sensors described above. Preferably, the second activity sensor 4 is a pneumatic pressure measuring device 4c.

According to a first embodiment, the occlusion section 6 comprises a balloon 8 that can be ovoid. This variation is shown in particular in FIG. 2.

The balloon 8 can comprise a force sensor 4a on its perimeter. Alternatively or in combination, the balloon can contain a pressure sensor 4b.

Preferably, the balloon 8 is linked to an external pressure measuring device 4c to measure the pneumatic pressure of the balloon 8.

The preferred variation of the first embodiment comprises:

a blocking balloon 8 of a diameter greater than 2 cm and less than 6 cm corresponding to a maximum dilation of the rectal ampulla;

a measurement balloon in the measurement section 7, approximately 3 cm in size and configured to be arranged in proximity to the blocking balloon 8, for example approximately 2 cm away. This measurement balloon is equipped with a photoplethysmographic sensor, in particular, the sensor is inside the balloon but facing the wall;

a preferably hollow catheter body 1a; and

an acquisition and processing unit that analyzes the pressure variations to indicate rectal activity.

The hollow body 1a of the catheter enables the passage of:

the cables 2a of the perfusion sensor 2;

an air inlet channel to inflate the measurement balloon 7; this air inlet is connected to a pressure sensor 4c; and

a second air inlet channel to inflate the semi-occlusive balloon 8. This air inlet is connected to a pressure sensor 4c.

According to a second embodiment, the occlusion section 6 comprises a container 9 and an evacuation channel 10. This variation is shown in particular in FIG. 3. Thus, according to a second embodiment, the occlusion section is in the form of a container such as a cone shape extending along said evacuation channel 10. Advantageously, the evacuation channel enables the evacuation of the liquid stools and can thus limit the pressure of the stools on the occlusion section 6.

The container 9 can be placed in the deployed position by means of a stent, by an inflatable device such as a balloon or by a shape-memory device such as a shape-memory plastic.

The container 9 preferably comprises a force sensor 4a on its surface, in particular on the stent or on the shape-memory device. Alternatively or in combination, the container can comprise a pressure sensor inside the balloon, if necessary. In this case, the balloon can be a toric balloon.

The preferred variation of the second embodiment comprises:

a shape-memory funnel with several lips, with a maximum diameter greater than 3 cm and less than 4 cm; the part with the greatest diameter is equipped with a force sensor 4a to provide an axial stress measurement;

a measurement balloon on the measurement section 7, of approximately 3 cm in size, configured to be arranged in proximity, for example 2 cm away from the smallest part of the funnel 9. This measurement balloon 7 is equipped with a photoplethysmographic sensor 2. In particular, the sensor is inside the balloon but facing the wall;

a preferably hollow catheter body 1a; and

an acquisition and processing unit 5 that analyzes the pressure variations in order to indicate rectal activity.

The hollow body 1a of the catheter enables the passage of:

the cables 2a of the photoplethysmographic sensor;

the cables 4d of the force sensor;

an air intake channel to inflate the measurement balloon. This air inlet is connected to a pressure sensor 4c.

The invention also relates to an apparatus for monitoring the perfusion of a tissue of an organ, comprising a device 1 as described previously and a processing unit 5 linked to said sensors 2, 4.

The invention also concerns a method for monitoring the perfusion of a tissue of an organ comprising steps for:
acquiring tissue perfusion measurement data,
acquiring muscle activity measurement data of the organ 3,
monitoring the perfusion measurement data by taking into account the muscle activity data.

Preferably, the step of monitoring the perfusion measurement data by taking into account the muscle activity data includes monitoring outside periods of muscle activity, particularly beyond a threshold.

The invention also relates to a computer program that can be loaded onto a processing unit, comprising portions of program code for performing the steps of the method as previously described, when said program is run on said processing unit.

The measurement data are monitored over time. The experiment details such as the perfusion or force measurement units and the calibration of the instruments are known to a person skilled in the art or are disclosed in the above-mentioned international applications.

1. A device for monitoring the perfusion of a tissue of an organ, comprising
a perfusion sensor configured to detect a perfusion of the tissue, and
at least one activity sensor configured to detect a muscle activity of the organ, said at least one activity sensor being arranged in proximity to the perfusion sensor, said sensors being configured to be linked to a processing unit.

2. The device, according to claim 1, comprising an occlusion section comprising the activity sensor, and a perfusion measurement section comprising the perfusion sensor.

3. The device, according to claim 2, wherein the occlusion section is movable between a folded position in which it is small in a radial direction and a deployed position in which it is large in the radial direction.

4. The device, according to claim 3, wherein the occlusion section comprises a balloon, a stent or a shape-memory material, configured to cause the occlusion section to switch between the folded position and the deployed position.

5. The device, according to claim 2, wherein the perfusion-measurement section is inflatable and is movable between a folded position in which it is small in a radial direction and a deployed position in which it is large in the radial direction.

6. The device, according to claim 1, wherein the perfusion sensor is a photoplethysmographic sensor.

7. The device, according to claim 1, wherein the activity sensor is a pressure sensor, a force sensor and/or a pneumatic pressure-measurement device.

8. The device, according to claim 2, wherein the perfusion measurement section comprises a second muscle activity sensor.

9. The device, according to claim 8, wherein the occlusion section comprises a container and an evacuation channel.

10. An apparatus for monitoring the perfusion of a tissue of an organ, comprising:
a monitoring device, according to claim 1; and
a processing unit linked to said sensors.

11. The method for monitoring the perfusion of a tissue of an organ, comprising steps of:
acquiring tissue perfusion measurement data,
acquiring muscle activity measurement data of the organ,
monitoring the perfusion measurement data by taking into account the muscle activity data.

12. The method for monitoring perfusion according to claim 11, wherein the step of monitoring the perfusion measurement data by taking into account the muscle activity data includes monitoring outside the muscle activity periods, particularly beyond a threshold.

13. A computer program that can be loaded onto a processing unit, comprising portions of program code for performing the steps of the method according to claim 10, when said program is run on said processing unit.
